(19) United States
(12) Patent Application Publication
(10) Pub. No.: US 2016/0024164 A1
(43) Pub. Date: Jan. 28, 2016

(54) CHEMOTHERAPEUTIC AGENTS COMPRISING A CHIMERIC GENE, METHODS OF PRODUCING THE SAME, AND METHODS OF TREATING CANCEROUS CELLS USING THE SAME

(71) Applicant: Michelle Yvette Andrzejak, Royal Oak, MI (US)

(72) Inventor: Michelle Yvette Andrzejak, Royal Oak, MI (US)

(21) Appl. No.: 14/806,586

(22) Filed: Jul. 22, 2015

Related U.S. Application Data
(60) Provisional application No. 62/027,306, filed on Jul. 22, 2014.

(51) Int. Cl. C07K 14/47

(52) U.S. Cl. CPC C07K 14/4747; A61K 38/00

(57) ABSTRACT

Chemotherapeutic agents, methods of producing the chemotherapeutic agents, and methods of treating cancerous cells using the chemotherapeutic agents are provided herein. In an embodiment, a chemotherapeutic agent includes a chimeric gene comprising an anti-apoptotic Bcl-2 family promoter region and a pro-apoptotic Bcl-2 family coding region. A method of producing the chemotherapeutic agent includes isolating an anti-apoptotic Bcl-2 family gene and isolating a pro-apoptotic Bcl-2 family gene. An anti-apoptotic Bcl-2 family promoter region is cleaved from the anti-apoptotic Bcl-2 family gene and a pro-apoptotic Bcl-2 family coding region is cleaved from the pro-apoptotic Bcl-2 family gene. The anti-apoptotic Bcl-2 family promoter region and the pro-apoptotic Bcl-2 family coding region are ligated to form the chimeric gene. A method of treating cancerous cells includes introducing the chimeric gene into the nucleus of the cancerous cells.

### CROSS-REFERENCE TO RELATED APPLICATIONS

This application claims the benefit of U.S. Provisional Application No. 62/027,306, filed Jul. 22, 2014.

### TECHNICAL FIELD

The technical field generally relates to chemotherapeutic agents that include a chimeric gene, methods of producing the same, and methods of treating cancerous cells using the chemotherapeutic agent. More particularly, the technical field relates to chemotherapeutic agents that include a chimeric gene that targets cancerous cells that exhibit abnormal cell cycle regulation, over-expressed proteins within the cancerous cells, or other gene products produced thereby, methods of producing the chimeric genes, and methods of treating cancerous cells using the chimeric genes.

### BACKGROUND

Techniques for treating various cancerous cells vary greatly and rely upon various different mechanisms. Cancerous cells generally exhibit abnormal cell cycle regulation, often resulting in unregulated proliferation, and such abnormalities in the regulation of the cell cycle are often targeted by specific treatments. For example, various chemotherapies rely upon targeted termination of fast-growing cells. Other chemotherapies specifically rely upon targeting cancerous cells based upon the presence of certain proteins or over/under-expression of certain proteins. However, such techniques are often accompanied by undesirable, visible side-effects such as hair loss, thinning of the skin, ulcerations to the gastrointestinal lining, etc. Further, such techniques are often of limited effectiveness due to damage to normal tissue and administration complications. Such techniques also often become less effective as the targeted cancerous cells develop alternative ways to evade the therapy.

It is known that cancerous cells often over-express certain genes, resulting in over-production of proteins or other gene products (e.g., RNA) that are regulated by the over-expressed genes. Such over-expressed genes are generally known as oncogenes. Presently, efforts are being made to target the over-expressed proteins or gene products that are produced by oncogenes by developing inhibitors to inhibit the function of the proteins or other gene products, thereby negating the over-expression of the subject proteins or other gene products. Such efforts generally focus upon inhibiting or negating the effect of the protein that is over-expressed. For example, antibodies; competitive inhibitors; other proteins that interfere with binding of a transcription factor protein to its target; and growth factor analogues can function as inhibitors to prevent the subject proteins from binding to their target substrates (e.g., DNA, other proteins, etc). Existing agents inhibit the function of the subject proteins or other gene products, thereby sequestering or inactivating the over-expressed proteins but not solving the root problem, which is continued proliferation of the cancerous cells when the over-expressed proteins do actually bind to their target. Thus, such efforts are hindered by the tendency of cells to stall proliferation due to the inhibition function while later finding ways to bypass the inhibited pathway.

Accordingly, it is desirable to develop alternative treatments to target cancerous cells, particularly, treatments that target oncogenes, over-expressed proteins, and/or other gene products produced thereby. In addition, it is desirable to develop alternative treatments that are difficult to bypass. Additionally, it is desirable to identify alternative treatments that may exhibit less severe bystander effects on normal cells than existing treatments. Furthermore, other desirable features and characteristics of the present invention will become apparent from the subsequent detailed description, taken in conjunction with this background.

### BRIEF SUMMARY

Chemotherapeutic agents, methods of producing the chemotherapeutic agents, and methods of treating cancerous cells using the chemotherapeutic agents are provided herein. In an embodiment, a chemotherapeutic agent includes a chimeric gene comprising an anti-apoptotic Bcl-2 family promoter region and a pro-apoptotic Bcl-2 family coding region.

In another embodiment, a method of treating cancerous cells includes introducing a chimeric gene into the nucleus of the cancerous cells. The chimeric gene includes an anti-apoptotic Bcl-2 family promoter region and a pro-apoptotic Bcl-2 family coding region.

In another embodiment, a method of producing a chemotherapeutic agent includes isolating an anti-apoptotic Bcl-2 family gene and isolating a pro-apoptotic Bcl-2 family gene. An anti-apoptotic Bcl-2 family promoter region is cleaved from the anti-apoptotic Bcl-2 family gene and a pro-apoptotic Bcl-2 family coding region is cleaved from the pro-apoptotic Bcl-2 family gene. The anti-apoptotic Bcl-2 family promoter region and the pro-apoptotic Bcl-2 family coding region are ligated to form a chimeric gene.

### DETAILED DESCRIPTION

The following detailed description is merely exemplary in nature and is not intended to limit the disclosure or the application and uses of the chemotherapeutic agents, methods of producing the chemotherapeutic agents, or methods of treating cancerous cells as described herein. Furthermore, there is no intention to be bound by any theory presented in the preceding background or the following detailed description.

Chemotherapeutic agents, methods of producing the chemotherapeutic agents, and methods of treating cancerous cells using the chemotherapeutic agents are provided herein. In particular, chemotherapeutic agents are proposed that may target cancerous or over-proliferative cells over normal cells using over-expressed protein activity that is characteristic of many cancerous cells to activate the chemotherapeutic agent, leading to cell death. Without being bound by theory, it is believed that over-expressed proteins (e.g., Myc, Lsf, E2f, and NFkB) or other gene products often act as transcription factors and bind to regulatory regions of a targeted gene, leading to transcription and expression of the coding/product sequence of the targeted gene. Over-expression of proteins and other gene products may occur through various mechanisms. As such, the genes that are targeted by the over-expressed proteins or other gene products are over-produced in many different types of cancers due to binding of the over-expressed proteins with the regulatory regions of those genes.

The chemotherapeutic agents described herein include a chimeric gene and, optionally, a delivery vector for delivering the chimeric gene into the nucleus of targeted cancerous cells. The chimeric gene includes an anti-apoptotic Bcl-2 family promoter region and a pro-apoptotic Bcl-2 family coding region, with the chimeric gene under the transcriptional control of the anti-apoptotic Bcl-2 family promoter region. Chimeric genes, also known as recombinant DNA, are non-naturally occurring polynucleotides that include the ligated product of genetic material from multiple sources. Instead of binding to oncogenes in cancerous cells and inhibiting protein over-production by the oncogenes, the chimeric genes described herein exploit activity of the oncogenes, namely over-expression of anti-apoptotic Bcl-2 family genes as is known to occur within certain types of cancerous cells, to cause cell death (not cell survival and proliferation). Activity of the oncogene is exploited by relying upon over-expressed transcription factors that ordinarily initiate over-expression of anti-apoptotic Bcl-2 family genes to instead bind with the anti-apoptotic Bcl-2 family promoter region of the chimeric gene and cause transcription of the pro-apoptotic Bcl-2 family coding region, which causes disruption to organelles such as mitochondria or endoplasmic reticulum and results in regulated cell death or apoptosis. Upon introduction of the chimeric gene into a cell, it is believed that the over-expressed transcription factors and/or other gene products of the cell that would otherwise target the anti-apoptotic Bcl-2 family genes will also target the regulatory region of the chimeric gene to thereby cause transcription of the coding region of the pro-apoptotic Bcl-2 family coding region in the chimeric gene, leading to transcription and apoptosis while also competing with the endogenous targeted genes for binding with the over-expressed protein or other gene product. Because anti-apoptotic Bcl-2 family genes regulate apoptosis downstream of many other regulatory signals in the cell signaling pathway, it is believed that the targeting mechanisms of the proposed chemotherapeutic agents will be difficult to bypass. Additionally, it is believe that cancerous cells that over-express anti-apoptotic Bcl-2 family genes will be more vulnerable to apoptosis as a result of exposure to the chemotherapeutic agents as described herein compared to normal cells, thereby potentially leading to therapeutic treatments that exhibit less severe bystander effects on normal cells than existing treatments.

As alluded to above, the chemotherapeutic agent includes the chimeric gene that includes the anti-apoptotic Bcl-2 family promoter region and the pro-apoptotic Bcl-2 family coding region. As referred to herein, “anti-apoptotic Bcl-2 family promotor regions” are promoter regions that include a BH1, BH2, BH3, and BH4 domain. Various anti-apoptotic Bcl-2 family promoter regions are targeted by proteins or transcription factors that are often over-expressed in various cancers such as but not limited to, e.g., STAT, Myc, Lsf, E2f, or NFkB. In embodiments, the anti-apoptotic Bcl-2 family promoter region is chosen from Bcl-2 or Bcl-xL, which in particular are targeted by Myc or NFkB. Additionally, matrix metalloproteinases (MMPs) are also targeted by certain proteins that are over-expressed in other cancers such as, e.g., Lsf or NFkB. In other embodiments, the chimeric gene includes a MMP promoter region as an alternative to the anti-apoptotic Bcl-2 family promoter region. As also referred to herein, “pro-apoptotic Bcl-2 family coding regions” are coding regions that include a BH3 domain and are free from a BH4 domain. In embodiments, the pro-apoptotic Bcl-2 family coding region only includes a BH3 domain. In other embodiments, the pro-apoptotic Bcl-2 family coding region includes BH1, BH2, and BH3 domains. In embodiments, the pro-apoptotic Bcl-2 family coding region is chosen from Bax or Bak. In specific embodiments, the anti-apoptotic Bcl-2 family promoter region is chosen from Bcl-2 or Bcl-xL and the pro-apoptotic Bcl-2 family coding region is chosen from Bax or Bak.

In embodiments, the anti-apoptotic Bcl-2 family promoter region and the pro-apoptotic Bcl-2 family coding region are linked together through a linking region. For example, the linking region may include complementary bases from an anti-apoptotic Bcl-2 family promoter region segment and a pro-apoptotic Bcl-2 family coding region segment that are isolated through conventional recombinant DNA formation techniques as described in further detail below. In particular, the linking region may include the reaction product of unpaired nucleotides, or “sticky ends”, that flank the promoter region and the coding region in the respective segments and that can associate to link the respective segments together. It is to be appreciated that while the segments are initially linked through the sticky ends (which generally exhibit binding through hydrogen bonds), further interactions may also occur within the chimeric gene to strengthen the bond between the two segments. For example, DNA ligase may be employed to form a covalent bond between a sugar-phosphate residue of adjacent nucleotides to join the two sugar-phosphate residues together. In other embodiments, the respective segments may include “blunt ends” instead of the sticky ends and the linking region may include the ligated product of the blunt ends. However, the chimeric genes as described herein are not limited to a particular linkage configuration. In embodiments, the linking region is free from additional functional regions such that the anti-apoptotic Bcl-2 family promoter region and the pro-apoptotic Bcl-2 family coding region are essentially linked directly together through the sticky ends or the blunt ends that form the linking region.

In embodiments, the chimeric gene is free from additional promoter regions other than anti-apoptotic Bcl-2 family promoter regions. Further, in embodiments, the chimeric gene is also free from additional coding regions other than pro-apoptotic coding regions. In this regard, the chimeric gene may consist essentially of the anti-apoptotic Bcl-2 family promoter region and the pro-apoptotic coding region as functional portions thereof, with the linking region provided primarily for linking the anti-apoptotic Bcl-2 family promoter region. In other embodiments, the chimeric gene may further include another functional region, such as a marker region, a metabolic region, or a combination thereof. For example, in embodiments, the chimeric gene may further include a marker segment linked to the pro-apoptotic Bcl-2 family coding region, on an opposite side thereof from the anti-apoptotic Bcl-2 family promoter region, to provide a signaling mechanism indicating expression of the chimeric gene. Specific examples of marker segments include, but are not limited to, a green fluorescent protein (GFP) region, a luciferase reporter region or a LacZ reporter region. As another example, in other embodiments, the chimeric gene may further include a metabolic segment linked to the pro-apoptotic Bcl-2 family coding region, on an opposite side thereof from the anti-apoptotic Bcl-2 family promoter region, to enhance the efficiency of the transcript.

An exemplary method of producing the chemotherapeutic agent described above includes producing the chimeric gene. To produce the chimeric gene, conventional recombinant DNA formation techniques may be employed. For example, in embodiments, an anti-apoptotic Bcl-2 family gene is isolated and a pro-apoptotic Bcl-2 family gene is isolated by extracting DNA from human cells, running the extract on a gel, and identifying the genes of interest through probe hybridization. The DNA may then be extracted from the gel, verified, and amplified. An anti-apoptotic Bcl-2 family promoter region is cleaved from the anti-apoptotic Bcl-2 family gene and a pro-apoptotic Bcl-2 family coding region is cleaved from the pro-apoptotic Bcl-2 family gene. The anti-apoptotic Bcl-2 family promoter region and the pro-apoptotic Bcl-2 family coding region are ligated to form a chimeric gene. In embodiments, the anti-apoptotic Bcl-2 family promoter region and the pro-apoptotic Bcl-2 family coding region are inserted into separate plasmids. In various embodiments, the anti-apoptotic Bcl-2 family gene promoter region and the pro-apoptotic Bcl-2 family coding region are replicated either prior to insertion into the plasmids, or after insertion into the plasmids whereby the plasmids are cultured to replicate and maintain the anti-apoptotic Bcl-2 family gene promoter region and the pro-apoptotic Bcl-2 family coding region. Anti-apoptotic Bcl-2 family gene promoter segments and pro-apoptotic Bcl-2 family coding segments are then cleaved from the plasmids using restriction enzymes to produce segments having the sticky ends. The anti-apoptotic Bcl-2 family promoter region and the pro-apoptotic Bcl-2 family coding region are then ligated to form a chimeric gene. More particularly, the respective segments that include the anti-apoptotic Bcl-2 family promoter region and the pro-apoptotic Bcl-2 family coding region are ligated through the sticky ends to form the chimeric gene.

As alluded to above, the chemotherapeutic agent further includes a delivery vector with the chimeric gene contained within or bound to the delivery vector. Conventional or proprietary delivery vectors may be employed. In embodiments, the delivery vector is chosen from oncoretroviruses, lentiviruses, or adenoviruses that are modified for gene delivery. Various such delivery vectors are known in the art and are commercially available. For example, suitable delivery vectors are commercially available as adenoviral or lentiviral vector systems from Clontech Laboratories, Inc. of Mountain View, Calif. In embodiments, the delivery vector targets specific cell types and such delivery vectors have been described in the literature. See, e.g., Yang et al., “Targeting lentiviral vectors to specific cell types in vivo,” PNAS, vol. 103, no. 31, p. 11479-11484, Aug. 1, 2006, the disclosure of which is incorporated herein by reference in its entirety. For example, a delivery vector that targets tissue of a particular cancerous cell of concern may be chosen to target treatment with the chimeric genes described herein to provide more targeted delivery and to minimize any potential bystander effect on non-targeted tissues.

In embodiments, the chemotherapeutic agent as described above may be included in a composition along with additional components. For example, the chemotherapeutic agent may be combined with conventional aqueous or non-aqueous carriers to enable controlled delivery of the chemotherapeutic agent. It is also to be appreciated that chemotherapeutic agent may be incorporated into other therapeutic formulations as a component thereof.

In embodiments, methods of producing the chemotherapeutic agent as described herein may include producing the chimeric gene in accordance with the techniques described above. However, it is to be appreciated that in other embodiments, the chimeric gene may be obtained from a third party and need not necessarily be produced in accordance with the methods of producing the chemotherapeutic agent as described herein. In embodiments, the chimeric gene is introduced into the delivery vector with the resulting chemotherapeutic agent including the chimeric gene in the delivery vector.

In embodiments, methods of treating cancerous cells using the chemotherapeutic agents as described herein include introducing the chimeric gene that includes the anti-apoptotic Bcl-2 family promoter region and the pro-apoptotic Bcl-2 family coding region into the nucleus of the cancerous cells. In embodiments, the chimeric gene is introduced into cells in a manner that results in entry into the nucleus of the cells. As set forth above, the chimeric gene may be introduced into the nucleus of the cancerous cells by delivering the chimeric gene into the nucleus of the cancerous cells using the delivery vector. In another embodiment, transformation may be employed, by which exogenous DNA is incorporated into endogenous DNA, to directly introduce the chimeric gene into cancerous cells without use of the delivery vector. Transformation may be employed, for example, by directly injecting the chimeric gene into a tumor. In another embodiment, electroporation may be employed whereby a cancerous cell is exposed to the chimeric gene followed by a pulse of electricity to enable uptake of the chimeric gene. In various embodiments, the chimeric gene may be introduced into the nucleus of the cancerous cells through ex vivo or in vivo introduction techniques.

It is to be appreciated that the chimeric gene may effectively function in the same manner for all cells into which the chimeric gene is introduced. In normal, non-cancerous cells, there is a strong synergistic balance between various proteins. However, because the over-expressed proteins in cancerous cells result in an imbalance between the various proteins, it is believed that the chimeric gene is more likely to be expressed in the cancerous cells and that the later effects of transcription and apoptosis will be triggered at a greater rate than for normal cells. Accordingly, it is believed that dosages of the chimeric gene can be set to differentiate between normal cells and cancerous cells by initiating transcription and apoptosis at a greater rate within the cancerous cells that contain over-expressed proteins or other gene products as compared to the normal cells, thus leading to apoptosis of the cancerous cells while allowing the normal cells to survive. Delivery mechanisms that target introduction of the chimeric gene into specific cell types cells (e.g., certain viral delivery mechanisms), as described above, can also be employed to minimize bystander effect on the normal cells.

In embodiments, methods of treating cancerous cells further include identifying a defect in cell cycle regulation of candidate cancerous cells to be treated. For example, the defect in cell cycle regulation may be over-production of Bcl-2 in the candidate cancerous cells to be treated and detection of the over-production of Bcl-2 in the candidate cancerous cells may be conducted in accordance with the treatment methods described herein. In other embodiments, the defect in cell cycle regulation may be over-production of a protein that targets the anti-apoptotic Bcl-2 family promoter region in the candidate cancerous cells to be treated and detection of the over-production of the subject proteins in the candidate cancerous cells may be conducted in accordance with the treatment methods described herein. For example, over-production of a protein chosen from STAT, Myc, Lsf, E2f, and/or NFkB may be detected in the candidate cancerous cells to be treated. After detecting the aforementioned defects in cell cycle regulation, the chimeric gene is introduced into the cancerous cells that exhibit the detected defect.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A chemotherapeutic agent comprising:
a chimeric gene comprising an anti-apoptotic Bcl-2 family promoter region and a pro-apoptotic Bcl-2 family coding region.

2. The chemotherapeutic agent of claim 1, wherein the anti-apoptotic Bcl-2 family promoter region is chosen from Bcl-2 or Bcl-xL.

3. The chemotherapeutic agent of claim 1, wherein the pro-apoptotic Bcl-2 family coding region is chosen from Bax or Bak.

4. The chemotherapeutic agent of claim 1, wherein the anti-apoptotic Bcl-2 family promoter region is chosen from Bcl-2 or Bcl-xL and wherein the pro-apoptotic Bcl-2 family coding region is chosen from Bax or Bak.

5. The chemotherapeutic agent of claim 1, wherein the anti-apoptotic Bcl-2 family promoter region and the pro-apoptotic Bcl-2 family coding region are linked together through a linking region comprising complementary bases from an anti-apoptotic Bcl-2 family promoter region segment and a pro-apoptotic Bcl-2 family coding region segment.

6. The chemotherapeutic agent of claim 1, wherein the chimeric gene is free from additional promoter regions other than Bcl-2 family promoter regions.

7. The chemotherapeutic agent of claim 1, wherein the chimeric gene further comprises a marker region.

8. The chemotherapeutic agent of claim 1, further comprising a delivery vector wherein the chimeric gene is contained within or bound to the delivery vector.

9. The chemotherapeutic agent of claim 8, wherein the delivery vector targets specific cell types.

10. The chemotherapeutic agent of claim 9, wherein the delivery vector is chosen from oncoretroviruses, lentiviruses, or adenoviruses.

11. A method of treating cancerous cells, wherein the method comprises:
introducing a chimeric gene comprising an anti-apoptotic Bcl-2 family promoter region and a pro-apoptotic Bcl-2 family coding region into the nucleus of the cancerous cells.

12. The method of claim 11, wherein introducing the chimeric gene into the nucleus of the cancerous cells comprises delivering the chimeric gene into the nucleus of the cancerous cells using a delivery vector.

13. The method of claim 11, wherein introducing the chimeric gene wherein introducing the chimeric gene into the nucleus of the cancerous cells comprises introducing the chimeric gene into the nucleus of the cancerous cells through an ex vivo introduction technique.

14. The method of claim 11, wherein introducing the chimeric gene into the nucleus of the cancerous cells comprises introducing the chimeric gene into the nucleus of the cancerous cells through an in vivo introduction technique within a mammal.

15. The method of claim 11, further comprises identifying a defect in cell cycle regulation of candidate cancerous cells to be treated.

16. The method of claim 15, wherein identifying the defect comprises identifying over-production of Bcl-2 in the candidate cancerous cells to be treated.

17. The method of claim 15, wherein identifying the defect comprises identifying over-production of a protein that targets the anti-apoptotic Bcl-2 family promoter region in the candidate cancerous cells to be treated.

18. The method of claim 17, wherein identifying over-production of the protein that targets the anti-apoptotic Bcl-2 family promoter region comprises identifying over-production of the protein chosen from STAT, Myc, Lsf, E2f, and/or NFkB in the candidate cancerous cells to be treated.

19. A method of producing a chemotherapeutic agent, wherein the method comprises:
isolating an anti-apoptotic Bcl-2 family gene;
isolating a pro-apoptotic Bcl-2 family gene;
cleaving an anti-apoptotic Bcl-2 family promoter region from the anti-apoptotic Bcl-2 family gene and a pro-apoptotic Bcl-2 family coding region from the pro-apoptotic Bcl-2 family gene;
ligating the anti-apoptotic Bcl-2 family promoter region and the pro-apoptotic Bcl-2 family coding region to form a chimeric gene.

20. The method of claim 19, further comprising introducing the chimeric gene into a delivery vector.
